(19) United States
(12) Patent Application Publication
(10) Pub. No.: US 2014/0046199 A1
(43) Pub. Date: Feb. 13, 2014

(54) METHOD FOR MEASURING HUMAN VITAL SIGNS AND PORTABLE TERMINAL ADOPTING THE SAME

(71) Applicant: Samsung Electronics Co., Ltd., Gyeonggi-do (KR)

(72) Inventor: Lei WANG, Hubei Province (CN)

(73) Assignee: Samsung Electronics Co., Ltd., Gyeonggi-do (KR)

(21) Appl. No.: 13/958,839

(22) Filed: Aug. 5, 2013

(30) Foreign Application Priority Data
Aug. 9, 2012 (CN) 201210281605.3

(51) Int. Cl. A61B 5/0205

(52) U.S. Cl. CPC A61B 5/02055
USPC 600/483

(57) ABSTRACT

A method for measuring human vital signs and a portable terminal adopting the same provides accurate measurements without increasing the thickness of power usage of the portable terminal. The portable terminal includes: a surface acoustic wave (SAW) sensor module disposed on a predetermined position of a portable terminal main body to detect characteristic parameter of human body; a signal transceiving module used to perform communication with the SAW sensor module, to receive an echo signal of the SAW sensor module; and a data processing module used to process the echo signal from the SAW sensor module to obtain the human vital signs. The SAW sensor module includes a sensing unit that does not require receiving power from a power supply for operation when implemented as a passive element.

### CLAIM OF PRIORITY

This application claims the benefit under 35 U.S.C. §119(a) from a Chinese patent application filed on Aug. 9, 2012 in the State Intellectual Property Office and assigned Serial No. 201210281605.3, the entire disclosure of which is hereby incorporated by reference.

### BACKGROUND

1. Field of the Invention

The present disclosure relates to a method for measuring human vital signs. More particularly, the present invention relates to a method for measuring human vital signs by measuring certain characteristic parameters such as body temperature, blood pressure, pulse or the like, in real time, as an additional accessory of a portable terminal, and a portable terminal adopting the same.

2. Description of the Related Art

Currently, the measurement of human vital signs such as body temperature and blood pressure is mostly performed in hospital or at home with discrete devices, and mostly by mercurial thermometer and sphygmomanometer. However, the mercury used in a thermometer is poisonous and such devices are vulnerable to breakage. With regard to a sphygmomanometer, such devices are not easy to carry. Accordingly, there is a need for a new technology and device that can replace these discrete devices used for measuring human vital signs.

With the development of the medical technology in recent years, people began to use an electronic thermometer and an electronic sphygmomanometer. When measuring the body temperature, an infrared ray based wireless body temperature measurement method and an active electronic sensor based method can be used. An active electronic sensor method is becoming more popular for measuring the blood pressure as it does not require pumping a squeeze-bulb. However, an apparatus adopting such solutions may require an extra power supply, increasing both weight and volume. In addition, such an apparatus also lacks portability and functions thereof as the device is still relatively simple.

Thus, there is a need in the art for a method and a portable device capable of measuring human vital signs in real time.

### SUMMARY

The present disclosure provides a method and apparatus for measuring human vital signs in real time using SAW elements and measures a plurality of human vital signs at the same time.

In addition, the present disclosure provides a portable terminal having a function for measuring human vital signs, which measures human vital signs in real time by using a surface acoustic wave (SAW) elements disposed on an accessory, such that the portable terminal has a small size and is easy to carry, which can also measure a plurality of human vital signs virtually any time and anywhere.

According to the present disclosure, a portable terminal with a function for measuring human vital signs is provided, in which the portable terminal comprises: a surface acoustic wave (SAW) sensor module for detecting characteristic parameters of a human body; a signal transceiving module (main body transceiver) for performing communication with the SAW sensor module, to receive an echo signal of the SAW sensor module; and a data processing module receiving and processing the echo signal from the SAW sensor module to obtain the human vital signs.

The signal transceiving module may transmit, for example, a query signal to the SAW sensor module to perform matching verification, which is performed for ascertaining a particular device, not a particular user.

The SAW sensor module may be located on an additional accessory of the portable terminal.

The additional accessory on which the SAW sensor module may be located may include, for example, one of a stylus pen, earphones and a data transmission cable of the portable terminal.

The characteristic parameter of a human vital sign that is measured may include at least one of temperature and pressure.

The portable terminal may include a notification module used to provide a measurement result of one or more human vital signs to be provided to a user as at least one of a display or a notification text, email, audio alarm, visual alarm, and the portable terminal can also store the measurement result and forward same to healthcare personnel, such as doctor, nurse/nurse practitioner, pharmacist, designated healthcare proxy, parent, sibling, friend, etc.

The SAW sensor module may include: a sensing unit having a SAW sensor without requiring a power supply for operation to sense/detect the characteristic parameter of a human vital sign; a data modulation unit for modulating the characteristic parameter as the echo signal; and a signal transmission unit (SAW Sensor Module Transceiver) for receiving signals from the portable terminal main body and transmitting the echo signal to the portable terminal main body. The SAW module can have structure that does not include a separate power supply for example, and through, for example, the piezoelectric effect, the SAW module is operational for its intended purpose.

The SAW sensor module may include a main body recognition unit for correlation in matching and/or recognizing on a main body a tag code included in a query signal from the signal transceiving module.

The data processing module may include an accessory recognition unit for performing a matching verification on an accessory identification tag (ID-tag) included in the received echo signal.

The accessory recognition unit may perform the matching verification through an application, wherein, the application includes a recognition code corresponding to the ID-tag of the SAW sensor module.

The data processing module may obtain human signs by performing an operation using at least one of an amplitude, a phase, a frequency and a time delay of the echo signal.

According to another aspect of the present disclosure, a method for operating an electronic device comprising a surface acoustic wave (SAW) sensor module comprises: detecting a characteristic parameter of a human body through the SAW sensor module and modulating the detected characteristic parameter of the human body as an echo signal; transmitting the echo signal to the portable terminal main body; demodulating the echo signal to obtain human vital signs through the main body of portable terminal. The sensed/detected characteristic parameter of the human body can include vital signs that for example include but are not limited to temperature, blood pressure, pulse, body mass index (BMI), glucose levels, etc., just to name a few non-limiting possibilities.

The electronic device may include handheld device, such as a portable terminal, including but in no way limited to smartphone, tablets, mini-tablets, phablets media players.

The SAW sensor module may be provided as an accessory of the portable terminal, for example, that is within the housing of the portable terminal, or is a discrete accessory or embodied within a discrete accessory such as a stylus pen, earphones, or other type of item worn by the user of the portable device.

The additional accessory may include one of a stylus pen, earphones and data transmission cable of the portable terminal.

The characteristic parameter of the human body being measured may include at least one of temperature and pressure.

The detecting a characteristic parameter of a human body may include: performing corresponding matching recognition on a query signal from the portable terminal main body; detecting the characteristic parameter if the recognition result matches with a main ID tag stored; and modulating the characteristic parameter and including the modulated characteristic parameter into the echo signal.

A portable terminal includes a surface acoustic wave (SAW) sensor module configured for detecting one or more characteristic parameters of human body utilizing acoustic waves and outputting an echo signal based on the detected one or more characteristic parameters; a main body transceiver that performs communication with the SAW sensor module, and receives the echo signal output by the SAW sensor module; and a data processor configured for receiving and processing the echo signal output from the SAW sensor module to obtain human vital signs, wherein the obtained vital signs are provided to at least one of user or a remote recipient.

The method may further include an application having a recognition code corresponding to an ID tag of the sensor module.

The demodulating of the echo signal to obtain human vital signs through the main body of portable terminal may include: performing matching verification on an accessory ID tag included in the received echo signal; demodulating the echo signal if the verification result matches with a stored accessory ID-tag, and performing operation using at least one of an amplitude, a phase, a frequency and a time delay of the echo signal to obtain human vital signs.

The matching verification can be performed through an application executed by hardware wherein, the application includes a recognition code corresponding to the ID-tag of the SAW sensor module.

The method may further include a notification function for providing a measurement result to a user by a visual display or audio, and can include notification of a remote entity, including but not limited to doctor, nurse, hospital, health care advocate, guardian, relative, etc.

According to present disclosure, a non-mercury measurement is implemented using the surface acoustic technology and a passive SAW sensor can measure a plurality of parameters such as temperature and blood pressure at the same time. In addition, through the combination with a mobile terminal, the detection apparatus can be miniaturized, thus the parameters of human body such as temperature, blood pressure and pulse can be measured conveniently and in real time.

### BRIEF DESCRIPTION OF THE DRAWINGS

FIG. 1 is a block diagram of a system for measuring human vital signs provided by the present disclosure.

FIG. 2 is a block diagram of a surface acoustic wave (SAW) sensor module of FIG. 1.

FIG. 3 is a portable terminal main body of FIG. 1.

FIG. 4 is an illustrative view of demodulation signal.

FIG. 5 is a flowchart illustrating operation of a method for measuring human vital signs according to the present disclosure.

FIG. 6 is a flowchart illustrating operation of a method for measuring human vital signs according to the present disclosure applied to a mobile phone.

### DETAILED DESCRIPTION

The terms “unit” or “module” as used herein is to be understood as constituting structure that operates in conjunction with hardware such as a circuit, integrated circuit, processor or microprocessor configured for a certain desired functionality in accordance with statutory subject matter under 35 U.S.C. §101 and definition of such terms do not constitute software per se.

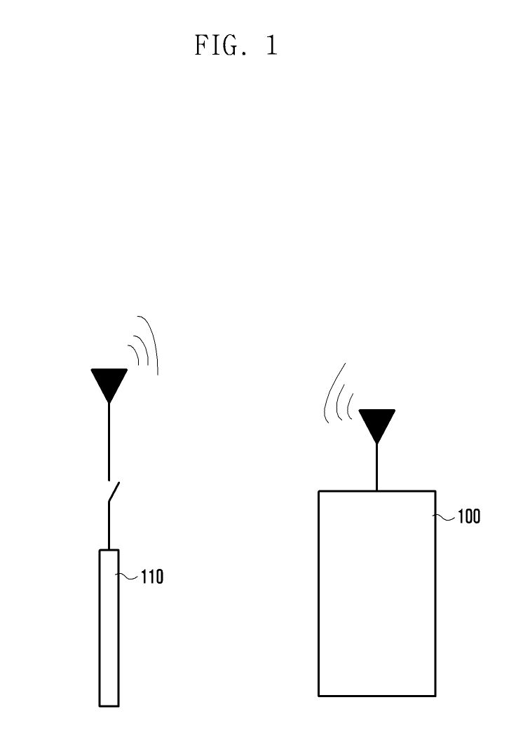

Hereinafter, the present disclosure is described in detail with reference to the drawings. FIG. 1 is a block diagram of a human vital signs measuring system provided by the present disclosure.

With reference to FIG. 1, the system for measuring human vital signs includes a surface acoustic wave (SAW) sensor module 110 and a main body 100. In this particular non-limiting example, the main body 100 may comprise a portable terminal main body 100. The SAW sensor module 110 may perform data communication with the portable terminal main body 100 in a wireless and/or wire manner, even though the drawing shows an antenna attached to both the SAW sensor module 110 and main body 100.

As commonly known, the surface acoustic wave (SAW) technology is a technology used in an apparatus for measuring acceleration, stress, strain, temperature, pressure and/or other parameters. Generally, a SAW element may be mounted on a piezoelectric substrate, the piezoelectric substrate can include but is not limited to, for example, quartz, lithium niobate, lithium tantalite, langasite optical crystal, or the like. The SAW element may generally include a pair or a plurality pairs of inter digital structure capable of converting the applied electrical signal into the electrical surface acoustic wave signal. Thus, all of the external parameters causing the variation of the stress and temperatures of the SAW element can be detected, and these variations can be measured in human vital signs. Such variation can be recognized by the piezoelectric effect, according to an offset of a resonant frequency in association with a SAW resonator or according a delay time or a phase offset of an electrical signal transmitted from a SAW delay line component.

The SAW sensor module 110 may be disposed on a predetermined position of the portable terminal main body 100 and is used to detect characteristic parameters of human body according to a query signal from the portable terminal main body 100. The predetermined position may be located at an additional accessory of the portable terminal, and may be located at any position on the portable terminal main body. The predetermined position may be any position conveniently contacted when the measurement performed.

According to the present disclosure, the portable terminal may be, for example, a mobile communication terminal (a hand phone), a notebook, a tablet PC, a personal digital assistant, a multimedia player, a navigator, or the like. The additional accessory may be, for example, a stylus pen (a handwriting pen), earphones, a wireless mouse, a data transmission cable, a probe, or the like. In this regard, when a detection is required, a part disposed with the SAW sensor module 110 is placed to the detection part (for example, an armpit, and so on) to perform sensing. Thus, when the SAW sensor module 110 is disposed on the additional accessory, a switching element may be equipped to turn on/turn off the SAW sensor module 110. In the following exemplary description, the portable terminal is in the form of a mobile communication terminal as an example, and the additional accessory is in the form of the stylus pen on the mobile communication terminal as an example. At this time, the SAW sensor module is disposed on a terminal of the stylus pen and a sensor antenna is integrated in the stylus pen.

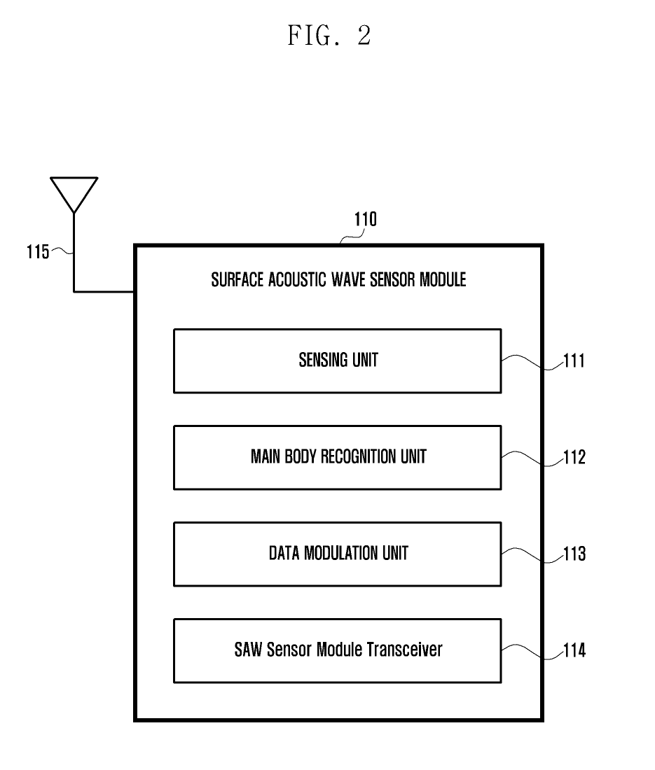

FIG. 2 is a block diagram of the SAW sensor module 110. The SAW sensor module 110 may transmit/receive data signal through an antenna 115, and may include a sensing unit 111, a main body recognition unit 112, a data modulation unit 113 and a signal transmission unit (SAW Sensor Module Transceiver) 114.

As shown in FIG. 2, the sensing unit 111 has a passive SAW sensor to detect characteristic parameters of the human body. As an example, some of the characteristic parameters of human body that can be measured may be a temperature and a pressure. Since the sensing unit 111 is implemented as a passive element, it is not required that power must be supplied from an external source, thereby facilitating use. The sensing unit 111 may include a plurality of SAW resonators connected in parallel on the piezoelectric substrate. Such SAW resonators may include inter-digital transducers for generating a surface acoustic wave in a different direction, such that the sensing unit 111 may measure a temperature, an acceleration and a pressure, or the like.

The sensing unit 111 has a tag coding function. Particularly, the tag coding function is implemented through the coding structure composed of the inter digital transducer and a reflecting grating. In other words, when the query signal from the portable terminal main body 100 is received, the inter digital transducer generates the surface acoustic wave, the surface acoustic wave is coded as a coded surface acoustic wave pulse sequence by the coding structure during transmission, and the coded surface acoustic wave pulse sequence is coded as a coded electrical pulse sequence signal through a positive piezoelectric effect, such that the tag coding function is implemented.

After detecting the characteristic parameters (pressure and/or temperature), the sensing unit 111 outputs the detected characteristic parameters to the data modulation unit 113, such that the data modulation unit 113 contains circuitry to modulate the characteristic parameters into the surface acoustic wave and the transmission characteristic of the surface acoustic wave (for example, a frequency shift, a phase shift, or the like) is changed, thereby the surface acoustic wave being modulated as an echo signal.

The Saw Sensor Module Transceiver 114 includes associated circuitry to transmit the echo signal modulated by the data modulation unit 113 to the portable terminal main body 100.

With continued reference to FIG. 2, the main body recognition unit 112 performs a correlation matching recognition on a main body tag code included in the query signal from the portable terminal main body 100. If the recognition result is a match, the data modulation unit 113 modulates an echo signal having the ID tag together with the characteristic parameters such as the temperature and the pressure. From this, after receiving the echo signal, the portable terminal main body 100 may firstly perform a matching verification of the ID tag, and maintain a state for receiving the echo signal in the case where there is no matching verification of the ID tag. If the tag information is matched, the echo signal is demodulated and a body temperature, a blood and a pulse data can be obtained by calculating according to one or more variations in an amplitude, a phase, a frequency, or a time delay of the echo signal. The example implementing method may be as follows: reference values of one or more of the amplitude, the phase, the frequency, or the time delay of the query signal are set when the portable terminal performs a measurement calibration, and the variation of the temperature and/or pressure information ambient to the sensing unit 111 may affect one or more of the amplitude, the phase, the frequency, the time delay of the echo signal to occur a sensed change of them. The portable terminal samples the echo signal, obtains the variation of the parameters between the query signal and the echo signal by comparing. There may be certain linear relationships between the above parameters (namely, for example, an amplitude, a phase, a frequency, a time delay of the echo signal) and the temperature and the pressure, respectively. Therefore, the body temperature and the blood pressure data can be obtained by detecting the absolute values of the above parameters. The above measurement process may be implemented through a measurement application installed in the portable terminal. Under the control of the measurement application, the measurement calibration may be completed first, and then the process of the actual measurement may be started. An artisan understands and appreciates that the frequency of calibration may be a periodic calibration, or even based on usage. The measurement of the body temperature is performed in accordance with a variation and stabilization process, in which the dynamic measurement is firstly displayed, and then the final measurement result is displayed. Of course, there can be various modifications, for example, where only the final measurement is displayed (and another screen is shown during the dynamic measurement, or a bar graph or percentage completion that is updated with progress. In the measurement of the blood pressure, the systolic pressure and the diastolic pressure of the blood pressure may be obtained by the calculation according to the amplitude and the period of the measurement waveform, and the pulse data information may be obtained according to the periods. Preferably, the actual measurement result may be displayed on the screen of the portable terminal (for example, a hand phone, smart phone, etc.) in a manner of comparing the actual measurement result and the normal value so the user can be informed as to whether his measurement is within a normal range or if action is required, and the final data may be stored into the portable terminal (for example, a storage module 150 of the portable terminal main body 100) or the cloud database.

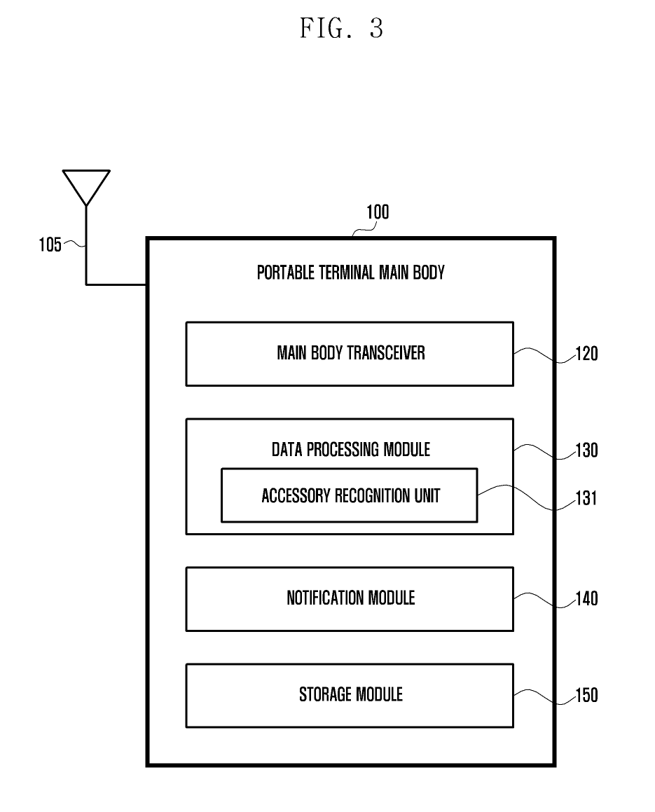

FIG. 3 is a block diagram of the portable terminal main body of FIG. 1. As illustrated in FIG. 3, the portable terminal main body 100 may include a signal transceiving module (main body transceiver) 120, a data processing module 130, a notification module 140, and a storage module 150.

The main body transceiver 120 includes circuitry such as a transceiver and is used to perform communication with the SAW sensor module 110, transmit a query signal to the SAW sensor module 110 and receive an echo signal from the SAW sensor module 110. The signal transceiving module 120 can be implemented as an antenna 105 on the portable terminal main body 100 (the antenna being shown schematically as extending therefrom, but an artisan understands and appreciates that most modern electronic devices normally do not extend a discrete telescopic antenna, particularly hand held electronic devices). An ID tag may be included in the query signal transmitted from the signal transceiving module 120 to the SAW sensor module 110, to perform a matching verification between the main body and the accessory.

The data processing module 130 includes hardware such as a processor or microprocessor that may be realized as an integrated circuit, processes the echo signal received by the signal transceiving module 120 to obtain the human vital signs. Particularly, the data processing module 130 may include an accessory recognition unit 131 comprising a comparator or sub-processor for performing the matching verification on the accessory ID tag included in the received echo signal. If the ID tag is matched, the echo signal is demodulated, and the values of the body temperature, the blood pressure and pulse, or the like can be obtained by calculating according to one or more parameters of the amplitude, the phase and/or the frequency and/or the time delay of the echo signal.

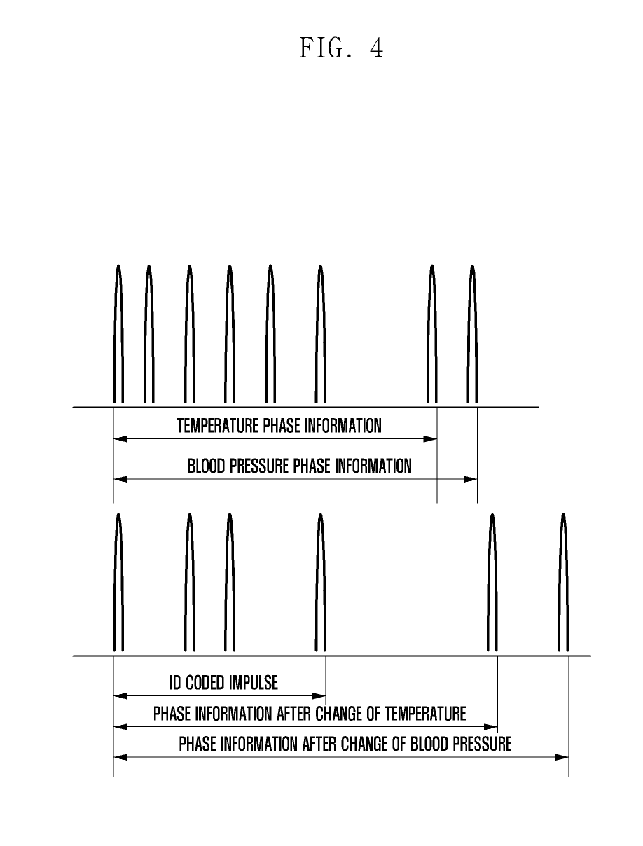

For example, as illustrated in FIG. 4, in the echo signal, a different accessory is indicated as a different impulse. Meanwhile, the temperature and the pressure information sensed at the accessory may change the phase of the sensing signal, thus providing data regarding the body temperature and the blood pressure, or the like of human body can be obtained by the demodulation of the phase information.

In addition, with continued reference to FIG. 3, the data processing module 130 may further include a reference value setting module for setting reference values of one or more parameters of the amplitude, the phase, the frequency, the time delay of the query signal, etc., when the portable terminal performs the measurement calibration. The reference values may be previously set according to the condition of the user' body, or can be automatically set when the measurement is performed. In addition, the accessory recognition unit 130 may include an application executed by hardware such as a processor having a recognition code corresponding to the ID tag of the SAW sensor module, thus the matching verification may be automatically performed through the application.

The notification module 140 is used to notify the measurement result to the user, the notification manner includes hardware associated with a voice notification and/or an image notification. For example, when the notification module 140 is a display module, the display module may display on a display screen the body temperature or the blood pressure data demodulated by the data processing module 130. Preferably, the display manner may be numeric, alpha-numeric or graphic. For example blood pressure data that indicates high blood pressure may be displayed differently from measurement data in a normal or typical range.

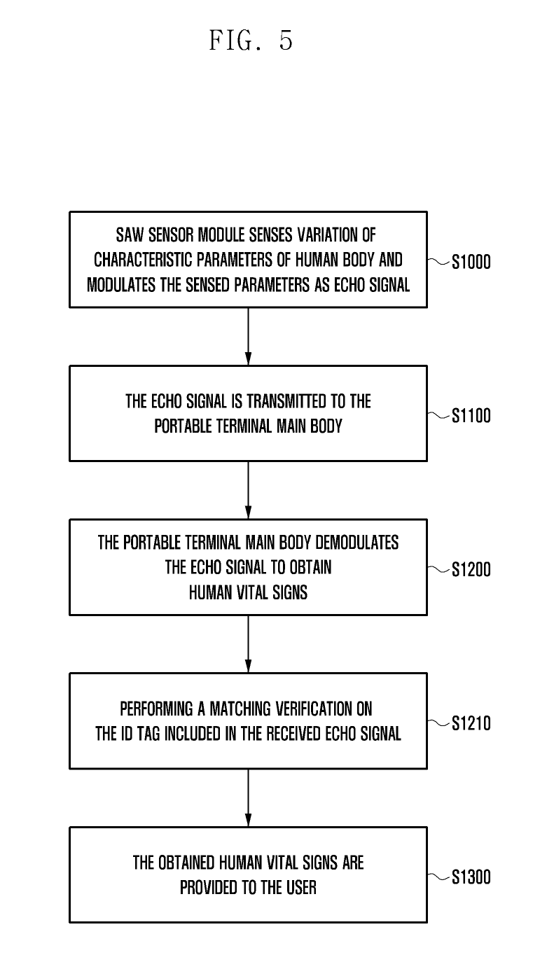

FIG. 5 is a flowchart illustrating exemplary operation of a method for measuring human vital signs.

Referring now to FIG. 5, at S1000, the SAW sensor module 110 senses a change of characteristic parameters of human body, and modulates the change of the characteristic parameters as an echo signal. The echo signal may include ID tag information and characteristic parameters such as temperature and pressure, or the like. Preferably, at S1000 the SAW sensor module 110 may perform a correlation matching recognition of a main body ID tag on a query signal from the portable terminal main body 100. If the recognition result is that the ID tag of the query signal matched with the stored ID tag, the SAW sensor module 110 detects characteristic parameters of human body. Then, the detected characteristic parameters are modulated and included into the echo signal. Next, the echo signal is transmitted to the portable terminal main body 100 S1100. Note that the query signal includes a main body tag code for the SAW sensor module to verify the main body. Also, the query signal is for requesting the characteristic parameters such as the temperature and the pressure. That is, a matching verification is performed for ascertaining a particular device, not a particular user.

At S1100, the SAW sensor module 110 transmits the echo signal to the portable terminal main body 100.

At S1200, the portable terminal main body 100 performs a demodulation process on the echo signal to obtain human vital signs. At S1200 may optionally include performing a matching verification on the ID tag included in the received echo signal S1210. At this time, preferable, the matching verification may be performed through an application having a recognition code corresponding to the tag of the SAW sensor module 110.

If the verification result of S1210 is that it is matched with the stored ID of the accessory, the echo signal is demodulated and the human vital signs can be obtained by performing calculation using one or more of the amplitude, the phase, the frequency and the time delay of the echo signal.

Next, at the step S1300, the obtained measurement result (i.e., human vital signs) is provided to the user in the voice manner or the display manner by the portable terminal main body 100. The obtained measurement result can be stored in a local storage (e.g., the storage module 150) or a cloud database.

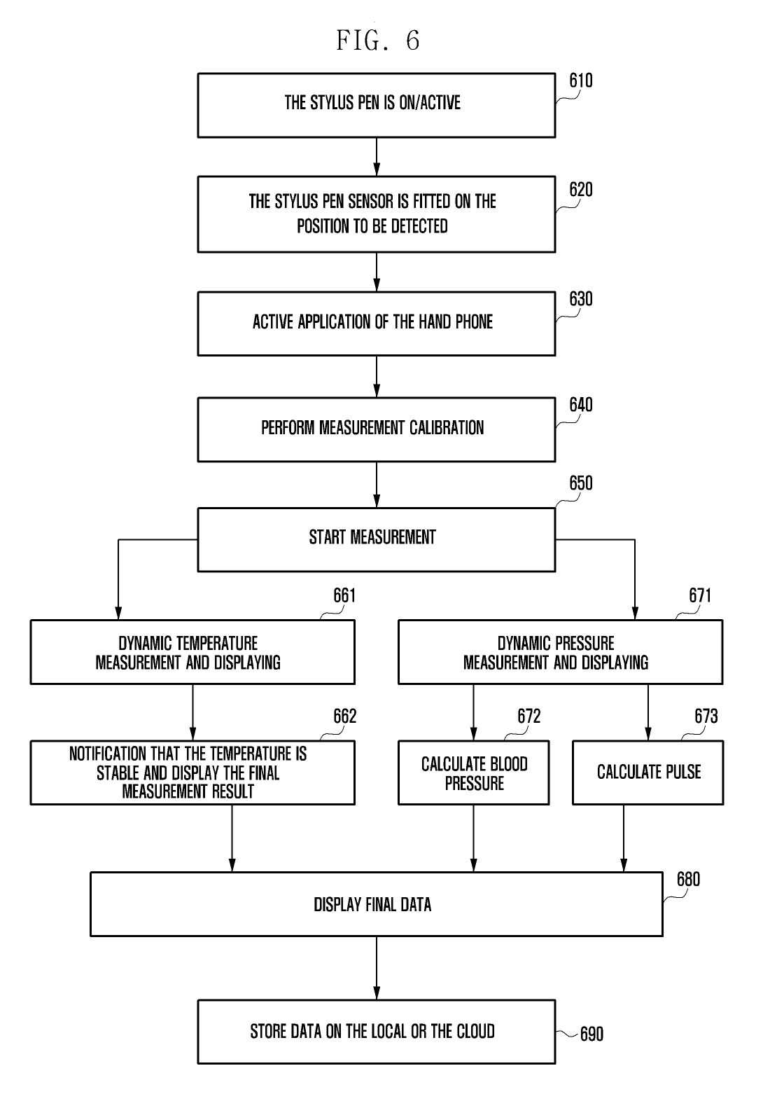

For better understanding, the following description on the detailed implementation manner of the present embodiment is made by taking a hand phone as an example. FIG. 6 is a flowchart illustrating exemplary operation of a method for measuring human vital signs according to the present disclosure applied to a hand phone (such as a handheld phone, smartphone, media player, etc. just to name a few non-limiting possibilities).

The SAW sensor module 110 may be integrated in a stylus pen of the hand phone, or may be integrated in an attached probe, or may be integrated with an antenna according to a shape of the stylus pen and be integrated with an appropriate passive matching circuit according to a desired capability. Other realizations of the SAW sensor module are possible.

At the hand phone side (e.g. portable communication terminal, except for being a main circuit of the hand phone, a transmitting circuit and a receiving circuit for a SAW sensor query signal, as a part of the hand phone, can also be integrated with a main board of the hand phone. Preferably, the transmitting circuit and the receiving circuit for the query signal may be an integrated circuit including a plurality of elements or a single chip.

With reference to FIG. 6, at 610 the stylus pen is on or active, at 620 and a sensing area of the stylus pen having the SAW sensor is fitted on the position to be detected. At 630, an application of the hand phone associated with the measurement such as body measurement and the blood pressure measurement, or the like, is activated.

At 640, under an automatic control of a processor, a measurement calibration is initially completed, which as discussed hereinbefore is not necessarily required for each measurement and may be performed periodically or based on accumulated usage.

At 650 an automatic measurement process is started. The automatic measurement process may include a temperature measurement process and a blood pressure measurement process. The only one of both processes may be performed, or alternatively both processes may be performed sequentially or simultaneously. In case of the temperature measurement process, the measurement of the body temperature needs a variation and stabilization process, the dynamical (or periodically progressive or interim) measurement is firstly displayed, and then the final measurement result is displayed.

More specifically, at 661 a dynamical temperature is measured, and when the temperature is stable, at 662 the user is notified and the final measurement result is displayed.

At 671, in the case of the blood pressure measurement process, a dynamical pressure is firstly measured and displayed.

At 672, that the systolic pressure and the diastolic pressure may be obtained by the calculating according to the amplitude and the period of the measurement waveform.

At 673, the pulse data information may be obtained/calculated according to a time period.

At 680, the actual measurement result may be displayed on the screen of the hand phone (an electronic device that may be handheld and including but not limited to a smartphone) in a manner of comparing the actual measurement result and the normal value.

At 690, the final data may be stored into the hand phone or via a cloud database. The results could also be emailed or sent as a text to a healthcare provider periodically or after a certain number of final data measurements. Meanwhile, the automatic measurement process may further include any other process (e.g., a pulse measurement process) associated with measurement of other vital signs.

The above-described embodiments according to the present disclosure can be implemented in hardware, firmware via the execution of software or computer code that is stored on a non-transitory machine readable medium such as a CD ROM, a RAM, a floppy disk, a hard disk, or a magneto-optical disk or computer code downloaded over a network originally stored on a remote recording medium or a non-transitory machine readable medium and stored on a local non-transitory recording medium, so that the methods described herein can be rendered via such software or computer code that is stored on the recording medium and executed by hardware such as a general purpose computer, or a special processor or in programmable or dedicated hardware, such as an ASIC or FPGA. As would be understood in the art, the computer, the processor, microprocessor controller or the programmable hardware include memory components, e.g., RAM, ROM, Flash, etc. that may store or receive software or computer code that when accessed and executed by the computer, processor or hardware implement the processing methods described herein. In addition, it would be recognized that when a general purpose computer accesses code for implementing the processing shown herein, the execution of the code transforms the general purpose computer into a special purpose computer for executing the processing shown herein. In addition, an artisan understands and appreciates that a “processor” or “microprocessor” constitutes hardware in the claimed invention. Under the broadest reasonable interpretation, the appended claims constitute statutory subject matter in compliance with 35 U.S.C. §101 and none of the elements constitute of software per se.

In the above description, the description is made reference with certain illustrative aspects of the present disclosure, however, the present disclosure is not limited to the disclosure shown and described herein. Those skilled in the art should understand that, various changes or modifications may be made to the illustrative description provided herein without departing from the spirit and scope of the disclosure as defined by the appended claims.

What is claimed is:

1. A portable terminal comprising:
a surface acoustic wave (SAW) sensor module configured for detecting one or more characteristic parameters of human body utilizing acoustic waves and outputting an echo signal based on the detected one or more characteristic parameters;
a main body transceiver that performs communication with the SAW sensor module, and receives the echo signal output by the SAW sensor module; and
a data processor configured for receiving and processing the echo signal output from the SAW sensor module to obtain human vital signs,
wherein the obtained vital signs are provided to at least one of user or a remote recipient.

2. The portable terminal of claim 1, wherein, the main body transceiver transmits a query signal to the SAW sensor module to perform matching verification.

3. The portable terminal of claim 1, wherein, the SAW sensor module is arranged on or in an accessory of the portable terminal.

4. The portable terminal of claim 3, wherein, the accessory includes one or more of a stylus pen, an earphone and a data transmission cable of the portable terminal.

5. The portable terminal of claim 1, wherein, the one or more characteristic parameters includes at least one of temperature and pressure.

6. The portable terminal of claim 1, wherein, further comprising a notification module used to provide a measurement result to a user of the portable terminal via at least one of audio or visual notification.

7. The portable terminal of claim 12, wherein the notification modules notifies the user or a designee via email or a text message.

8. The portable terminal of claim 1, wherein, the SAW sensor module comprises:
a sensing unit having a passive SAW sensor element that operates without receiving power from a power supply to detect the characteristic parameter;
a data modulation unit that modulates the one or more characteristic parameters as the echo signal;
a SAW sensor module transceiver that receives signals from the portable terminal main body and transmits the echo signal to the portable terminal.

9. The portable terminal of claim 1, wherein, the SAW sensor module further comprising a main body recognition unit for correlating matching recognizing of a main body tag code included in a query signal from the main body transceiver.

10. The portable terminal of claim 1, wherein, the data processing module further comprising an accessory recognition unit for performing a matching verification of a user on accessory identification tag (ID-tag) included in the received echo signal.

11. The portable terminal of claim 10, wherein, the accessory recognition unit performs the matching verification through an application executed by a processor of the data processing module, wherein, the application includes a recognition code corresponding to the ID-tag of the SAW sensor module.

12. The portable terminal of claim 10, wherein, the data processing module performs operation using at least one of an amplitude, a phase, a frequency and a time delay of the echo signal.

13. A method for operating a portable terminal comprising a surface acoustic wave (SAW) sensor module, comprising:
detecting by a sensor of the SAW sensor module one or more characteristic parameters of a human body and modulating the one or more detected characteristic parameters of the human body as an echo signal;
transmitting the echo signal to a main body of the portable terminal; and
demodulating through the main body of the portable terminal the echo signal to obtain at least one human vital sign.

14. The method of claim 13, wherein, the SAW sensor module is arranged on or in an accessory of the portable terminal.

15. The method of claim 14, wherein, the accessory includes at least one of a stylus pen, an earphone and a data transmission cable of the portable terminal.

16. The method of claim 13, wherein, the detected one or more characteristic parameters includes at least one of temperature and pressure of a living body.

17. The method of claim 13, wherein, the modulating the one or more detected characteristic parameters of the human body detected by the SAW sensor module comprises:
performing corresponding matching recognition of a user on a query signal from the portable terminal main body;
detecting the one or more characteristic parameters if the recognition result matches with a main ID tag stored; and
modulating the one or more characteristic parameters and including the modulated characteristic parameter into the echo signal.

18. The method of claim 13, wherein, the demodulating the echo signal by the SAW sensor module comprises:
performing matching verification on accessory ID tag included in the echo signal;
demodulating the echo signal when the verification result matches with a stored accessory ID-tag, and utilizing at least one of an amplitude, a phase, a frequency and a time delay of the echo signal to obtain human vital signs.

19. The method of claim 18, wherein, the matching verification is performed through an application executed by the data processor, wherein, the executed application includes a recognition code corresponding to the ID-tag of the SAW sensor module.

20. The method of claim 13, further comprising:
notifying a user by providing a measurement result of the at least one human vital sign by at least one of audio or visual notification.

21. A portable terminal including a stylus pen, comprising:
a sensor module located in the stylus pen and configured to detect one or more characteristic parameters of human body and output an echo signal based on the detected one or more characteristic parameters;
a main body transceiver configured to perform communication with the sensor module that receive the echo signal of the sensor module; and
a data processing module configured to process the echo signal received from the sensor module in order to obtain human vital sign.
